(19) United States
(12) Patent Application Publication
(10) Pub. No.: US 2010/0247489 A1
(43) Pub. Date: Sep. 30, 2010

(54) USE OF A COMPOSITION MADE OF MINERAL NUTRIENTS AND OPTIONALLY ACETOGENIC AND/OR BUTYROGENIC BACTERIA IN ORDER TO AVOID OR REDUCE THE FORMATION OF GAS IN THE LARGE INTESTINE OF A MAMMAL AND THE RESULTING ABDOMINAL PROBLEMS

(76) Inventor: Roland SAUR-BROSCH, Heidelberg (DE)

Correspondence Address: KEVIN J. MCNEELY, ESQ., 5335 WISCONSON AVENUE, NW, SUITE 440, WASHINGTON, DC, 20015, US

(21) Appl. No.: 12/520,561

(22) PCT Filed: Dec. 21, 2007
(86) PCT No.: PCT/EP07/11352

§ 371 Date: Dec. 15, 2009

(30) Foreign Application Priority Data
Dec. 22, 2006 (DE) 10 2006 062 250.2

(51) Int. Cl. A61K 35/74; C12N 1/20; A61K 33/24; A61K 33/04; A61P 1/00; C01B 19/02; C22C 28/00; C22C 27/04

(52) U.S. Cl. 424/93.4; 435/252.1; 424/617; 424/702; 424/646; 424/93.41; 424/93.48; 423/510; 420/579; 420/429; 420/430

(57) ABSTRACT

The present invention relates to a composition comprising one or more minerals selected from the group consisting of selenium, molybdenum or tungsten, which is carried out galenically or chemically in a way that the mineral or minerals are released completely or in part, just before, during or shortly after arrival at the large intestine, and their use in the manufacture of a medicament for administering to a mammal for the prevention or reduction of gas formation in the colon thus conditioned abdominal complaints, particularly bloatings, meteorism or abdominal cramps. Furthermore, the invention relates to a procedure for the isolation of acetogenic and butyrogenic bacterial strains that are suitable for therapeutic purposes outlined above.

Use of a combination of minerals and if applicable acetogenic and/or butyrogenic bacteria for the prevention or reduction of gas formation in the colon of a mammal, and thus induced abdominal complaints.

### DESCRIPTION

The present invention relates to a composition comprising one or more minerals selected from the group consisting of selenium, molybdenum or tungsten, which is carried out galenically or chemically in a way that the mineral or minerals are released in whole or in part, just before, during or shortly after the arrival at the colon, and their use in the manufacture of a medicament for administration to a mammal for the prevention or reduction of gas formation in the colon and thus induced abdominal complaints, particularly bloating, meteorism or abdominal cramps.

The invention further relates to a medicament for the prevention or reduction of gas formation in the colon of a mammal, and thus induced abdominal complaints, consisting of a composition comprising one or more minerals selected from the group consisting of selenium, molybdenum and tungsten or a combination thereof, and where required a pharmaceutically acceptable carrier.

Furthermore, the invention relates to a method for the isolation of acetogenic and butyrogenic bacterial strains that are suitable for the above-mentioned therapeutic purposes.

### TECHNICAL FIELD

The invention lies in the field of medicine and pharmacology.

### PRIOR ART

A large number of people are suffering from abdominal complaints, which are largely caused by unwanted metabolic products of microorganisms that colonize the human intestine. Among these metabolites particularly are ranking the gases built in the intestine (especially gases such as hydrogen, carbon dioxide, methane, hydrogen sulfide and nitrogen), as well as lactic acid, short chain fatty acids and various enterotoxins. Depending on the type and quantity of these metabolic products, differently marked symptoms arise for the affected people.

For instance the produced gases lead to symptoms such as abdominal bloating, meteorism, abdominal cramps and other secondary diseases diclining thereof.

Such sequelae include for instance, Small Intestine Bacterial Overgrowth Syndrome (SIBO) as a result of a disturbed function of the ileocecal valve caused by excessive colonic gas pressure.

Due to its osmotic effect, lactic acid formed by bacteria can cause diarrhea and may have pathological consequences in case of absorption by the colon mucosa.

Is a too high concentration of less efficient degradable D-(−)-lactic acid (ie, left-turning lactic acid) built by the intestinal bacteria, it can also lead to manifestation of lactic acidosis.

When absorbed into the bloodstream, enterotoxins can affect the nervous system.

For example, they can cause headaches, fatigue, irritability or even cause loss of consciousness. Furthermore, they can paralyze the muscles of the intestine and cause constipation. By irritation of the intestinal mucosa with subsequent release of fluid, the metabolic products of bacteria can also cause diarrhea. Bacterially formed alcohols are absorbed by the colon mucosa, and burden the liver, which can be damaged by the persistent alcohol load.

Such symptoms occur more often and at higher intensity when these metabolites arise in larger quantities, which is especially the case when the bacteria that produce these metabolites, are provided with amounts of nutrients beyond the normal. This is among others the case for malabsorbtions of carbohydrates such as by lactose malabsorption induced by lactase deficiency, fructosemalabsorption, malabsorption of sugar alcohols (eg sorbitol, xylitol, mannitol, lactitol, maltitol), disaccharase deficiency and trehalase deficiency, maldigestion, which trace back to exocrine pancreatic insufficiency, lack of gastric acid, bile acid deficiency or insufficient insalivation of chyme, celiac disease, sprue, gluten intolerance, for disorders of the masticatory apparatus, and also with increased intake of indigestible dietary components such as resistant starch or indigestible ingredients of cabbage and legumes.

After resection of the small intestine often partially malabsorptions/maldigestions occur due to reduced absorption area (short bowel syndrome).

Even at a quantitatively normal of gas production abdominal complaints may arise if the nerves in the intestinal tract response hypersensitively.

This is for instance the case if, like with the IBS, there is pain signalised already with a normal strain of the gut where a healthy human would only notice increased activity of the intestine.

In infants malabsorptions an maldigestions, which become discernible as so-called three-month colic, often occur due to the not entirely mature digestive system.

Furthermore intestinal complaints may occur frequently after antibiotic treatments, which probably is attributable to the alterations in the composition of the intestinal flora (eg dysbiosis induced by antibiotics).

Due to the lack of nutrient competition, some antibiotic-resistant bacteria such as Clostridium perfringens and Clostridium difficile, and yeast like Candida albicans propagate excessively and contribute to the complaints by their increasedly built metabolic products.

With maldigestion based on exocrine pancreatic insufficiency, lack of stomach acid or bile acid deficiency, good effective medicaments are usually available. However, in some cases, there still is not a complete elimination of symptoms, since both the dosage and the distribution in the chyme (eg, with the substitution of pancreatic enzymes) can not be done in such a way as to a proper organ function.

The treatment of malabsorption usually consists of a diet in which the malabsorbed nutrients are mostly or complete omitted. Under certain cercumstances this results in strong limitations to the choice of food and also does not lead to complete symtom relief, because certain nutrients, such as for example fructose are present in nearly all foods, at least in trace amounts.

In the case of lactose malabsorption it is also possible to administer the missing enzyme lactase in the form of powders, tablets or capsules, but this results in similar shortcomings regarding the dosage and distribution in the chyme, as with the substitution of pancreatic enzymes. In addition, these enzymes are partially extracted from fungal cultures, so that the final products are not tolerated for many allergy sufferers.

Another option for the treatment of lactose malabsorption consists of oral intake of lactose metabolising lactic acid bacteria (Lactobacillus spp. and Bifidobacterium spp.). The lactic acid bacteria will then metabolise a certain amount of malabsorbed lactose into lactic acid, so that a lower concentration of lactose arrives at the large intestine, and thus, less problematic metabolites arise there. If this metabolism takes place in the small intestine, a majority of the arisen lactic acid can be absorbed. The unabsorbed amount and the lactic acid produced only in the large intestine are further on able to be osmotically effective and cause diarrhea.

Since the transition time of the chyme through the small intestine is relatively short, during this time only a limited amount of lactose can be metabolized, so with this method an almost complete freedom from symptoms can be achieved only for quite low amounts of lactose.

Admittedly the administered bacteria also conduct in nutrient competition in the colon with the bacteria building problematic substances, but this rather takes place in a parallel metabolism, so that still significant amounts of the offending metabolites arise.

The administration of lactic acid bacteria (Lactobacillus spp. and Bifidobacterium spp.) is also used for other disorders than the lactose malabsorption, with the aim to displace the bacteria, which produce the problematic metabolites, in order to cause an allevation.

Problematic bacteria can not be completely eliminated by such displacement experiments because of the fact that certain bacteria can only increase to a certain maximum number per amount of intestinal content, because there are either not enough essential nutrients available or an inhibition takes place due to their own metabolic products.

Furthermore, lactic acid bacteria often have only a very limited rage of substrates, so that in most cases not all foods causing symptoms can be made harmless if the symptoms are caused by various indegestible carbohydrates.

With the so-called three-month colics the therapy for instance consists of administration of defoamers (e.g. simethicone) or the application of abdominal massages to encourage the faster exit of the bloating from the intestines as a flatus.

Also the administration oft lactic acid bacteria is recommended, however with the same restrictions regarding the efficacy as in the treatment of the abdominal disorders mentioned above.

In some cases it is tried to alleviate the symptoms by administration of antibiotics, with the aim to kill the bacteria, which are producing the metabolic products causing complaints.

The problem with this approach, however, is that indigestible nutrients still can arrive at the colon and there present a stimulus to the growth of bacteria that are able to utilize these substances.

In most cases the intestinal flora is left to itself after such antibiotic treatment, in the hope of a favorable bacteria constellation establishing by itselves, which is unfortunately not very often crowend with durable success.

Some therapists prescribe agents to stimulate the intestinal microflora, containing mainly lactic acid bacteria or non-pathogenic enterobacteria (Escherichia coli).

These can have a quite positive effect on the establishment of the intestinal flora by the acidification of the intestinal lumen and the occupation of the intestinal mucosa, but this will not constitute a purposeful way to ensure an unproblematic disposal of the gases formed by the bacteria.

By the application of the above-mentioned bacteria, the colonization of the desired acetogenic bacteria may even be prevented by the nutrients competition in certain circumstances.

Also with antibiotic therapies, which are not prescribed primarily for the elemination of certain bacteria of the intestinal flora, it is tried to obtain a positive reconstruction of the intestinal flora by the application of lactic acid bacteria or medical yeasts, which however show the above-described shortcomings.

Nollet L. et. al., Appl Microbiol Biotechnol, 1997, 48: 99 describe the effect of the addition of Peptostreptococcus productus in an artificial reactor system, which simulates the human intestine, on the intestinal flora and their activity.

The authors show that the production of methane by bacteria can be modified for the benefit of acetogenesis (ie, the conversion of carbon dioxide (CO2) and hydrogen (H2) to acetic acid) by the addition of this bacterial strain.

However, this effect decreases quite rapidly if the administration of the bacteria into the system is discontinued. Admittedly the acetogenic bacteria still continue to produce acetic acid, but primarily by heterotrophic homoacetogenesis and not by the desired hydrogenotrophic reductive acetogenesis.

Therefore, this approach to a pharmaceutical composition would not be satisfactory.

A bacterial strain with similar biochemical properties, Ruminococcus hydrogenotrophicus, is described by Bernalier A. et. al., Arch Microbiol, 1996, 166: 176. The strain was extracted from human feces and is capable of H2/CO2-conversion in vitro.

In the patent applications U.S. 2004/002 86 89 and WO 02/07741 non viable or attenuated pathogenic clostridia are administered with one or more other non-pathogenic or attenuated pathogenic microorganisms to influence the microflora in the intestine. The administration of acetogenic bacteria, with or without minerals/vitamins is however not mentioned.

Another approach is attempted in DE 102 06 995. In this print a prebiotic containing micronutrient combination product is described, which can be used as a dietary food for medical purposes. The combination product contains bacteria such as Bifidobacterium, Lactobacillus, Enterococcus, or other probiotic cultures, together with other vitamins and minerals. The combination product for example is intended to be used with allergies, chemotherapy and radiotherapy, gastroenteritis, chronic inflammatory bowel disease, or with lactose intolerance. From the fact that most people with gastrointestinal disorders, which cause malabsorption, have no or only minor complaints, and these people in contrast to symptomatical malabsorbers show higher amounts of acetic acid in the feces (Born et al.), it can be concluded that mammals with a sufficient reductive acetogenic metabolic activity of their intestinal bacterial flora have no or only minor complaints with malabsorptions and that in mammals with which abdominal complaints occur after the consumption of food, which contain non-absorbable nutritional components, the reductive acetogenic metabolic capacity of the intestinal bacterial flora is reduced.

It is therefore the purpose of the present invention to provide a composition and a method which is suitable for the prevention or treatment of increased gas formation in the colon and thus induced abdominal discomfort or gastrointestinal disorders.

The solution to the purpose consists in the provisioning of a composition comprising one or more minerals selected from the group consisting of selenium, molybdenum and tungsten or a combination thereof, and if applicable a pharmaceutically acceptable carrier.

This composition is usable for the prevention or treatment of increased gas formation in the colon and consequent abdominal complaints such as for example bloating, meteorism, abdominal cramps, three month colic, irritated bowel syndrome, irritable bowel, or for the treatment of gastrointestinal disorders which can lead to gas formation in the colon. Further included by the invention are medicines or dietary supplements, which include the aforementioned minerals, and are used for the reduction or prevention of the formation of gas in the colon.

Advantageous embodiments of the present invention will be apparent from the dependent claims.

### DESCRIPTION OF THE INVENTION

The invention is based on the surprising discovery that selenium, molybdenum and tungsten, in particular, alone or any combination, effect a significant reduction or prevention of gas formation in the colon of a mammal. Such colon targeted effectiveness of the aforementioned minerals for the treatment and prevention of abdominal complaints caused by increased gas formation in vivo is new and surprising.

The term “mineral or minerals,” as used here, especially the minerals selenium, molybdenum and tungsten, claimed in this invention include natural substances, basic substances, chemically modified variants thereof, compounds, derivatives and analogues of the respective mineral. Examples are sodium tungstate, sodium selenite, sodium selenate, selenomethionine, sodium, selenium, tungsten phytate, selenium phytate, tungsten oxalate, tungsten tannine.

By means of the mineral composition according to this invention, a significant reduction in the amount of gas in the intestine can be achieved, which leads to a significant improvement in the complaints of the aformentioned gastrointestinal disorders.

In certain cases it may be necessary, that one or more acetogenic or butyrogenic bacteria strains are administered to the mammal besides the claimed minerals, simultaneously, sequentially or as a component of the composition.

Due to the effect of the claimed minerals, the population and/or metabolic activity of the bacteria is promoted in a way that the number and/or quantity of the administered bacteria can be kept as low as possible and/or the frequency of administration can be reduced.

This variety is part of a preferred embodiment.

One advantage of the invention is to make it possible to also treat complaints which are not caused primarily by the increased rise of intestinal gases, but by an osmotically caused increased amount of liquid in the intestine induced by malabsorbed nutrient components.

As a result of several major symptoms of these gastrointestinal disorders which often occur in combination can be treated simultaneously by a single combined composition.

The inventor has noticed that the ability for reproduction of acetogenic bacteria, responsible for the gas reduction in the intestine of a mammal (such as the colon of a human) mostly depends on how well supplied the microorganisms are with energy sources (eg useable carbohydrates), and cell materials (eg nitrogen) and with additional co-factors (eg vitamins and minerals).

The inventor of the present invention particularly found out that the desired metabolic activity of individual bacteria on the other hand depends on in which concentrations they can be provided for with certain minerals, and with which activity the required enzymes for the various metabolic processes can be built.

Even with a constant re-supply of acetogenic bacteria, it is incedental that the bacteria may increase in count during the intestinal passage, the reductive acetogenic metabolic activity of the entire acetogenic bacteria may however not increase significantly, as because oft a lack of the needed minerals, the total amount of required enzymes cannot be increased significantly. Furthermore, a sufficient supply of the colonic acetogenic bacteria with the recurred minerals is not possible on a normal diet because the required heavy metals are presumably already absorbed to a large extent in the small intestine by the intestinal mucosa, and thus are no longer available in the colon in a sufficient quantity for the acetogenic bacteria.

Also a targeted increase of the according minerals in the diet is not the solution of the problem, because they are toxic in higher concentrations in the host organism.

This is the point, where the present invention is effective, in which the disadvantages of known processes and bacterial strains are effectively overcome.

On the one hand it influences the number of acetogenic bacteria and the balance of the population of the existing acetogenic bacteria in the intestinal flora. On the other hand it promotes the metabolic pathways of acetogenic bacteria, which are necessary for the prevention of the build-up of gas.

The invention proposes a pharmaceutically active composition for the avoidance of abdominal complaints, which includes the heavy metals selenium, molybdenum and/or tungsten, either alone or in any combination and is preferably prepared in a galenic form, such that the active ingredients are to be released only shortly before, at the time of or shortly after arrival at the large intestine.

Thereby the availability of the heavy metal ions for the acetogenic bacteria in the intestine is significantly increased while the percentage of heavy metals absorbed by the host organism is greatly reduced, thereby ensuring the supply of acetogenic bacteria without exposing the host organism to a high dose of heavy metals.

For cases in which the acetogenic bacteria get along with lower mineral concentrations and these are achieved with administration forms that are not designed for a preferable exclusively release in the colon without exposing the host organism to an acute risk of poisoning, the invention also proposes a composition which is not coated resistant against digestion in the small intestine.

According to the invention the minerals selenium, tungsten and molybdenum have proven advantageous for the propagation and promotion of acetogenic bacteria and their reductively acetogenic metabolic performance in the colon and thus for the reduction and avoidance of the formation of gas. The minerals may be contained in the composition individually or in any combination, either as a pure substance, natural substance, chemical compound, derivative or analog. For example, the minerals can be modified so that their release and efficacy in the colon is promoted. This can be achieved by binding the mineral to a chemical compound.

In a preferred embodiment, the invention additionally contains vanadium, nickel, iron, sodium or potassium, either individually or in any combination, even with the aforementioned minerals.

In addition, the following vitamins have proven advantageous for the reproductive and metabolic activity of acetogenic bacteria: vitamin A, vitamin B, vitamin C, vitamin D, vitamin E, vitamin K.

Preferably, minerals and vitamins should be included in one form of administration in the composition. Depending on the condition of the intestinal flora and disease, the composition additionally should contain one or more strains of acetogenic or if applicable butyrogenic bacteria and/or enterobacteriaceae.

The preferred form of administration of the inventive composition and the hence produced medicines or dietary supplements is an oral or rectal administration.

The galenic preparation for example may consist of a capsule or tablet coated resistant against digestion in the stomach and the small intestine, which's coating dissolves in the colon after a certain time after leaving the acidic stomach milieu.

Preferably, the composition and their active ingredients is continuously released in the colon over a period of 3 hours, preferably 6 hours, most preferably 12 hours. This time is intended to bridge the interval from one main meal to another. With daily take over several days, the desired effect is established (see sample 1 and 4).

Preferably, the galenic preparation consists of a capsule which's coating is dissolved by enzymes of colonic bacteria. For the galenic preparation of the invention, the further known techniques can be used in the present invention by the skilled person. By the use of a galenic preparation and targeted release in the colon the intestinal flora of acetogenic bacteria can be specifically developed or regenerated. Due to the addition of minerals and/or vitamins also the long-term conservation of the population and metabolic activity of these strains is ensured and thereby the success of the therapy, without the need for ongoing administration of viable acetogenic bacteria.

The admission of acetogenic bacteria may in fact prove to be disadvantageous in some cases, for example, when the body reacts susceptible to a specific (non autologous) strain or develops allergies. The admission of minerals or vitamins, however, leads to a specific stimulation of the autologous bacteria which are already present in the intestinal flora.

Even when using autologous strains of acetogenic bacteria to increase the population density in the intestine, it is advantageous to avoid an ongoing admission, because preparations with autologous strains are naturally extravagantly and expensively to manufacture because they must be prepared separately for each individual, and cannot be produced in large scale (as opposed to a mineral preparation).

Preferentially the minerals are present in the composition in a concentration of 1 ng to 3 mg per element and/or the vitamins at a concentration of 1 IU to 1000 IU (IU=International Unit) and/or acetogenic bacteria in a concentration of 1×105 to 1×1013 cfu (CFU=colony-forming unit) and/or the butyrogenen bacteria in a concentration of 1×10⁵ to 1×10¹³.

Another embodiment of the invention proposes that the needed minerals of the acetogenic bacteria are made available to the acetogenic bacteria as chemical compounds which can not or only slightly be absorbed in or by the intestine of the host. Thereby, they are available to the acetogenic bacteria, which can utilize the compounds for their own metabolism.

Furthermore, the compounds can be cleaved in the colon (eg, by enzymes from colonic bacteria) and the relieved minerals may be utilized by the acetogenic bacteria.

As suitable compounds have been proven mineral compounds with phytic acid, tannines, tannic acid or oxalic acid. For example phytate can be cleaved by bacterial phytases, whereby the bound minerals (such as selenium or tungsten) become available in the colon.

In a further embodiment of the invention it is proposed to administrate the minerals selenium, tungsten and/or molybdenum and possibly iron, individually or in any combination, in a galenic preparation, which is designed in a way that the mineral or the minerals are released delayed in time, preferably but not exclusively, with a evenly drug release over 48 hours.

Many galenic design options are available to the skilled person in the state of the art, of which some examples are also mentioned in this description.

Because the dwell time in the small intestine is approximately 3 hours, with a release period of 48 hours 6.25% of the minerals are already released in the small intestine and are absorbed into the bloodstream of the mammal by the mucosa of the small intestine.

This makes it possible, when using minerals which are salubrious for the mammal in small amounts, but toxic in larger amounts, that the treated mammal is supplied with low, physiological amounts of the physiologically precious mineral or minerals, but the larger amounts are available for the acetogenic bacteria in the colon.

Preferably a release term of at least 3 hours is chosen, so at least a part of the minerals arrive at the colon.

Another embodiment of the invention proposes to feed specifically to the acetogenic bacteria nitrogen and/or energy suppliers to increase their growth. Preferably are nitrogen-containing compounds or carbohydrates that are absorbed only hardly or marginally by the intestine. Preferred are those compounds which are absorbable by the intestine to a maximum of 75%, preferably not more than 50% and most preferably to a maximum of 25% or less.

Examples of such compounds are urea, difructoseanhydride, rhamnose, whereby the invention should not be limited to these examples.

Urea is not absorbed by the human intestine, and is hence available in the large intestine, where it will be used by the acetogenic bacteria for the build up of amino acids, needed for the propagation.

Are acetogenic bacteria with an adequate metabolic potential already present in the colon of the treated individual, their population and metabolic activity can be increased to an extent that a sufficient therapeutic effect can be achieved, preferably without the addition of acetogenic bacteria which are cultured in vitro.

This has major advantages because non autologous strains are often difficult to settle in the colon, because they do not find certain environmental factors, to which they are dependent on, in the treated individual.

Furthermore the possibility can not be ruled out that non autologous strains cause pathogenic reactions in certain individuals, even if they are not known to be potentially pathogenic.

The administration of autologous strains which do not show these disadvantages is therefore another aspect of the present invention.

These strains can be isolated and cultivated for each individual that is to be treated by the use of the inventive process.

In some cases it may be necessary to administer in addition to the above-mentioned minerals and/or vitamins acetogenic bacteria, possibly in combination with other bacterial strains. For example this is necessary when the metabolic activity of the acetogenic bacteria in the colon does not show the required efficacy or metabolic processes of other intestinal bacteria inhibited the desired acetogenesis.

Preferably, the minerals and vitamins are therefore administered orally or rectally to the host with a hydrogenotrophic acetogenic bacterial strain as a combination product. Appropriate strains are preferably such ones that are able to metabolize fructose and/or lactose, or the malabsorbed carbohydrate of the individual to be treated. Especially bacteria from the genus Ruminococcus, Eubacterium and Clostridium have proved themselves suitable.

Especially preferred strains of the present invention are Ruminococcus hydrogenotrophicus, Ruminococcus productus and Clostridium coccoides, Clostridium formicoaceticum and Eubacterium limosum.

Because each bacterial strain has specific requirements to its environment and this environment is the large intestine of the host which has individually different environmental parameters such as pH, redox potential, bacterial toxins of competitive bacteria, the invention proposes as an advantageous embodiment, to administer several different strains of acetogenic bacteria to increase the probability that at least one strain in the intestine of the host is sufficiently able to survive and propagate.

A particular advantage of the combined administration of acetogenic bacteria and minerals that are needed for the formation of the required enzymes for the reductive acetogenesis, is that enough minerals are available in the intestine of the host during the numerical propagation of the acetogenic bacteria, so that the bacteria propagated by division not only have available the halved amount of enzymes, but can provide the full reductive acetogenic metabolic performance by means of synthesis of the required enzymes, and the power of reductive acetogenic metabolism of the acetogenic bacteria population increases significantly during the intestinal passage.

Thereby in the case of a necessary supply of acetogenic bacteria, the required quantity of supplied bacteria can be reduced significantly and so the costs associated with the treatment can be lowered.

Another advantage of the invention is the ensurance of the availability of selenium, tungsten and/or molybdenum in the large intestine of the mammal.

Only after the inventive minerals were administered in a form that largely prevented an absorption in the small intestine, an adequate reductive acetogenic metabolic activity could be retained even after discontinuation of acetogenic bacteria, cultured in a mineral-containing medium without exposing the host to the risk of chronic heavy metal poisoning (see Example 1, Phase H, I and K).

It was further noted that upon the entry of large quantities of malabsorbed or maldigested nutrients, the concentration of acetic acid built by the acetogenic bacteria can rise severely due to the reductive and/or heterotrophic acetogenesis, which may inhibit the metabolic performance of the acetogenic bacteria. Depending on the species, the acetogenic bacteria prozesses H2 and CO2 very efficiently up to a certain concentration of acetic acid, while the efficiency decreases significantly at higher acid concentrations due to the product inhibition.

It was therefore searched for a way to reduce the concentration of acetic acid, which could be inventively achieved by a simultaneous injection of butyrogenic bacteria or targeted stimulation of butyrogenic bacteria which are already present in the intestine. These bacteria metabolize a significant amount of the arising acetic acid into butyric acid, thus lowering the concentration of acetic acid, thereby increasing significantly the growth, as well as the metabolic activity of acetogenic bacteria.

It is therefore proposed in a further embodiment of the invention that the inventive composition also contains butyrogenic bacteria (such as Fusobacterium, Faecalibacterium, and Eubacterium, with or without vitamins or minerals). Particularly advantageous are strains that predominantly metabolize acetic acid to butyric acid. An especially advantageous example is Faecalibacterium prausnitzii.

For cases in which an administration of minerals and vitamins, and energy and nitrogen sources for the promotion of the acetogenic bacteria may be omitted, a composition is suggested that contains butyrogenic bacteria.

In a further embodiment of the present invention the composition contains minerals, vitamins and/or nitrogen donors, and if applicable additional energy- and carbon-sources (for example carbohydrates) useable by the butyrogenic bacteria. In this combination it is possible to promote the butyrogenic bacteria in their growth and metabolic activity and to achieve an optimal activity of the acetogenic bacteria population in the colon by the associated turn-over of acetic acid.

Preferably, the nitrogen compounds and carbohydrates should not or only slightly be absorbed by the intestine, which can be achieved either by the primarily non-digestible nature of these additives in the host organism or by using an appropriate galenic preparation.

The encouragement of a settlement or retention and regeneration of acetogenic bacteria or their metabolic activation in the colon has been exposed especially for the treatment and therapy of abdominal symptoms, as acknowledged at first mention.

The positive impact of the composition of the invention are however not limited only to the treatment of abdominal pain, they are also evident to the prophylactic use. With the inventive composition non human mammals such as dogs, cats, pigs, cattle, sheep, goat or other farm animals and humans can be treated.

The administration of the composition is preferably carried out orally with the use of the aforementioned capsules with coating resistant against digestion in the small intestine.

Alternatively, the composition may be administered rectally as a suppository or a hydro-colon therapy.

It has been shown, that with a high mineral level in the intestinal contents in some cases the minerals can be absorbed by the colonic mucosa, which increases the risk of toxicity and reduces their availability to the intestinal bacteria.

Therefore, the invention proposes in a preferred embodiment, that the release of the minerals is delayed, for example, as part of the matrix core of a tablet with a coating resistant against digestion in the small intestine, a capsule with a diffusion membrane, a capsule with a defined micro-orifice and osmotically-controlled release, or other suitable galenic preparations, and ensures, that the mineral levels are not too high at the beginning of the colon, and are not lowered too much during the passage of the intestinal contents through the colon, and thus a possibly optimal mineral level is always available.

This results in less unwanted absorption at the beginning of the colon and in a sufficiently high mineral level at the end of the intestinal passage to ensure an effective performance of reductive acetogenic metabolism of the acetogenic bacteria.

Preferrably abdominal complaints are treated or prevented which are or would be caused by the following gastrointestinal disorders: fructose malabsorption, fructose intolerance, lactose intolerance, irritable bowel syndrome (IBS), lack of disaccharase, lack of trehalase, short bowel syndrome, Crohn's disease, three month colic, exocrine pancreatic insufficiency, bile acid deficiency, stomach acid deficiency, dysbiosis of the intestinal flora caused by antibiotics, celiac disease, sprue, gluten intolerance, histamine intolerance.

During or after an antibiotic therapy, hydro-colon therapy and colonic lavages or gastrointestinal infections, for example, with enteroviruses or pathogenic enterobacteria, an administration of the compositions of the described invention is also recommended.

Especially with an antibiotic therapy it is ensured by the reconstruction of the intestinal flora disturbed by the antibiotic(s), that an adequate amount of acetogenic and/or butyrogenic bacteria settles to prevent or at least reduce abdominal discomfort after eating indigestible food components (eg fiber) or with malabsorbtions and maldigestions.

It is therefore intended to use the compositions based on this invention, also during and/or after antibiotic therapy to support the intestinal reconstruction.

A combination with conventional lactic acid bacteria such as lactobacilli, bifidobacteria, enterococci and non-pathogenic enterobacteria, such as selected strains of Escherichia coli is also possible and is to utilize in individual cases depending on the condition of the intestinal flora and the occurring symptoms.

When administered to affected females the composition of the invention preferably contains selenium and/or tungsten. Tungsten has proved to be especially advantageous for treatment and prophylaxis of abdominal complaints and disorders in humans. Selenium is naturally absorbed in the upper small intestine in humans and therefore can not be available (without external supply) for the acetogenic bacteria in the colon to eliminate or prevent the formation of gas. Furthermore it was noticed that tungsten is absorbed strongly by affected females, which is one reason why affected females very often complain about a more increased gas formation than male affected patients.

The invention also relates to the use of a butyrogenic bacterial strain for the treatment and/or prevention of abdominal complaints in a mammal, which are accompanied by a decreased reductive acetogenic metabolic performance. For these compositions, the above-described variants and embodiments for acetogenic bacteria are accordingly valid.

For a settlement of administered butyrogenic bacteria in the human intestine, the generation time with the metabolism of carbohydrates and acetic acid is very important. For the therapeutic success the metabolic capacity under the prevailing conditions has to be paid attention for, which can be measured biochemically with known methods via the acetic acid degradation capacity. Exemplary genera of butyrogenic tribes are: Fusobacterium, Faecalibacterium, Eubacterium and Clostridium.

To find such beneficial butyrogenic bacterial species or strains of bacteria it is proposed in a further embodiment of the invention to isolate them from human feces, because there butyrogenic autologous bacteria are present, which have the required ability to survive in the human intestine. In the micro flora of the intestine generally only such butyrogenic bacteria will survive, which possess the corresponding metabolic activities and have prevailed over other bacteria. In this way a natural selection of bacteria comes off with the desired metabolic capacity.

In a preferred embodiment with the inventive method a dilution series with the isolated butyrogenic bacteria in a suitable solvent is prepared and applied to an anaerobic growth medium that contains acetic acid as the sole energy source and an acetic acid indicator.

Preferably, the breeding of these isolated butyrogenic bacteria is carried out under anaerobic conditions, if applicable in a nitrogen atmosphere.

The stool samples preferably should come from people who exhibit an underlying cause of the symptoms, which however are completely or at least largely asymptomatic. According to Born et al. only about 40% of the fructose malabsorbers exhibit gastrointestinal symptoms.

The inventor suspects that the asymptomatic malabsorbers are equipped with sufficiently effective working acetogenic and/or butyrogenic bacteria, so that such asymptomatic malabsorbers preferably come into question as bacterial suppliers. This increases the probability of finding such butyrogenic bacteria that are adapted to the utilization of acetic acid that is produced by the acetogenic bacteria in those people, and can effectively reduce it into butyric acid.

Cultures, which are excelled by a short generation time (visible as large colonies of cells) and by a pronounced acetic acid consumption (response of indicator) are preferably isolated and characterized in the aftermath of their generation time for acetic acid metabolism.

Analog to the isolation of preferred butyrogenic strains it is proposed to isolate preferred acetogenic bacteria strains with appropriate procedures. In contrast to the isolation of butyrogenic strains CO2 and H2 in the gas phase of the medium are provided as the sole energy and carbon source. An acid indicator serves as an indicator that shows how effective the bacteria metabolize the offered gas into acetic acid. Among others, thymol blue, methyl orange, methyl red or phenolphthalein can serve as indicators.

With a section of the people who suffer from gastrointestinal disorders, the symptoms are not or only partly show in bloating caused by gas formation, but also or mainly by diarrhea or intestinal cramps, caused for example by the osmotic effect of undigested food components.

Due to the acetogenic bacteria also practicing heterotrophic metabolism, they can relieve the symptoms in these cases. Due to the higher amounts of liquids, which pass into the intestine through the osmotic action, the intestinal transit time, however, is often reduced to such an extent that the heterotrophic metabolism of the acetogenic bacteria is inadequate to metabolize the osmotically acting undigested food components quickly enough.

It is known that enterobacteria, mainly Escherichia coli can metabolize carbohydrates more quickly than the most occurring bacteria in the colonic flora, and so mitigate osmotic diarrhea, or may even prevent it. However, these bacteria metabolize their used foodsubstrate from the carbohydrates primarily into gases (H2 and CO2), which may in turn lead to complaints from bloating.

The invention furthermore proposes a preferred embodiment, in which Enterobacteriaceae are administered additionally, preferably of the species Escherichia coli, in order to have the undigested food particles metabolized into gases by the enteric bacteria, so that these gases then can be metabolized by the acetogenic bacteria to acetic acid, either to be absorbed by the intestinal mucosa, or to be available as a substrate for butyrogenic bacteria.

This design benefits from the gas-metabolizing effect of one of the inventive compositions described above, to treat or prevent the side effects of bloating and meteorism, resulting from gas formation by enterobacteria with a conventional treatment of diarrhea.

Preferably, the administration is carried out in a combined pharmaceutical preparation.

The following examples illustrate the invention, which should not be limited to these, in detail.

### EXAMPLES OF WAYS FOR THE REALIZATION OF THE INVENTION AND COMMERCIAL UTILIZATION

### Example 1

Subject: Western European, male, 35 years old, normal weight, fructose malabsorption with bloating as the dominant symptom.

Preparation: Manufacturing of the bacterial pellets

Cultivation medium: For the preculture a modified cooked meat medium was used (RACh acc.to Nollet et al.). In addition, 0.1 mmol of sodium selenite and 0.1 mmol of sodium tungstate were added. Furthermore 1% fructose was added.

The prereduced medium was poured into 15 ml Hungate tubes, covered with 80% H2 and 20% CO2 in the gas phase and the freeze-dried pellets of the bacterial strain of Ruminococcus hydrogenotrophicus DSM 10507 (Deutsche Sammlung von Mikroorganismen und Zellkulturen GmbH) were added. The preculture was incubated for 48 hours at 37° C. To the Hungate tubes determined for centrifugation, no meat particles but only the broth were added.

The medium was filled under an anaerobic atmosphere (N2) in 10 ml Hungate tubes flooded with 80% H2, 20% CO2 and incubated with 0.5 ml of the preculture.

The incubation was ensued at 37° C. for 36 hours. Thereafter, the bacteria were separated by centrifugation and freeze-dried. Subsequently the coating with Eudragit FS 30 D was carried out, also under anaerobic atmosphere.

Phase A:

Basic diet: Strictly poor in fructose (<1 g/day)

Used food: rice, rice cakes, rice flakes, turkey, eggs, milk, yoghurt, sugar-free white wheatbread, smoked salmon, yams, watered potatoes, rapeseed oil, salt and pepper.

Dietary Supplements: multivitamin (Multibionata Tropfen, Merck), folic acid (Ratiopharm), zinc (Zinkorot). With the help of 7 g of Psyllium seed husks twice a day and 5 g of polyethylene glycol 4000 the stool consistency was adjusted in a way, that defecation on a 40 Charrière intestinal tube was possible. The basic diet was observed for 4 weeks. During this time, the subject was free of complaints.

Phase B:

Diet with fructose load: To the above-described basic diet were added 10 g of fructose per day, each 2 g daily in each of the 5 meals stirred as a powder directly into the food. During phase B, the subject complained of severe symptoms (bloating and abdominal cramps). The diet with fructose load was carried out until the end of the experiment. Phase B lasted 10 days.

Phase C:

Application of acetogenic bacteria: The strain used was Ruminococcus hydrogenotrophicus DSM 10507 (Deutsche Sammlung von Mikroorganismen und Zellkulturen GmbH). Pellets coated with Eudragit FS 30 D were swallowed non-chewed during the main meals (three times daily one coated pellet). From the second day of Phase C, the subject noticed a significant reduction of the complaints, which persisted until the end of Phase C. Phase C was performed for 10 days.

Phase D:

Discontinuation of the acetogenic bacteria. Acetogenic bacteria were now not administered anymore, but only the continued diet with fructose load. From the third day of phase D on, the subject again complained of intense discomfort, on the seventh day it was therefore decided, to directly changed to phase E (phase D had also been planned for 10 days).

Phase E:

Application of acetogenic bacteria and minerals: The acetogenic bacteria were administered analog to phase C. With each main meal, 10 μg selenium in form of sodium selenite and 20 μg tungsten as sodium tungstate were stirred directly into the meal as a powder additionally. From the second day of phase E on, the subject again noted the significant reduction in symptoms that lasted again until the end of this phase. Phase E was performed for 10 days.

Phase F:

Discontinuation of acetogenic bacteria, thereby continuing the mineral application. With each main meal, 10 μg of selenium in the form of sodium selenite and 20 micrograms of tungsten as sodium tungstate as a powder were stirred directly into the meal. From the third day of phase F on, the subject again complained of intense discomfort. On the seventh day it was therefore decided, to re-supply acetogenic bacteria again (there were also 10 days planned for phase F).

Phase G:

Reapplication of acetogenic bacteria: Phase G was performed analog to phase C. The process of complains was accordingly. Phase G was performed for 14 days until phase H had been developed.

Phase H:

Application of acetogenic bacteria and minerals in a coating resistant against digestion in the small intestine: The acetogenic bacteria were administered analog to phase C. With each main meal, 10 μg selenium in form of sodium selenite and 20 μg tungsten as sodium tungstate were administered. The mineral salts were compressed together with methyl cellulose into tablets and coated with Eudragit FS 30 D too, but with a higher layer thickness in order to achieve not only gastric juice resistance, but due to the longer disintegration time of the thicker coating to release the mineral salts only in the colon. From the second day of phase H on, the subject noticed that the complaints, already significantly reduced by the application of the acetogenic bacteria, had even disappeared entirely. Phase H was performed for 10 days.

Phase I:

Discontinuation of acetogenic bacteria, thereby continuing the mineral application with a coating resistant against digestion in the small intestine.

Analogous to phase H, with each main meal, 10 μg selenium in form of sodium selenite and 20 μg tungsten as sodium tungstate were administered, in a coating resistant against digestion in the small intestine made of Eudragit FS 30 D. A recurrence of the symptoms was not observed. Phase I was performed for 10 days.

Phase J: Discontinuation of minerals. Analogous to phase B only the diet with fructose load was administered. From the fourth day the complaints re-occurred, from the eighth day with full intensity as in phase B and F.

Phase K: Mineral application with a coating resistant against digestion in the small intestine. Analogous to phase I, with each main meal, 10 μg of selenium in the form of sodium selenite and 20 μg tungsten as sodium tungstate with a coating resistant against digestion in the small intestine made of Eudragit FS 30 D were administered. From the second day on the complaints decreased and from the fifth day on the subject was practically free of complaints. Phase K was performed for 10 days.

Phase L: Administration of butyrogenic bacteria. Since the amount of gas in phase I still was significantly higher than in phase A it was searched for a way to increase the performance of the acetogenic bacteria again. Because the interspecies nutrient transfer can effect a stimulation of metabolism, butyrogenic bacteria, namely faecalibacterium prausnitzii, were administered in addition to the minerals coated resistant against digestion in the small intestine analogous to the acetogenic bacteria

A subjective improvement in symptoms was not established, however, the amount of the intestinal gases could be further reduced, suggesting that the application butyrogenic bacteria can increase the tolerable amount of malabsorbed carbohydrates.

Phase M: Administration of non-encapsulated minerals in large quantity.

In order to investigate whether the application of unencapsulated minerals in higher doses (to compensate for the absorption in the small intestine) is effective, non-encapsulated mineral were administered again as powder, but the amount of minerals was increased tenfold per meal in contrast to the non-successful phases E+F, now per meal 100 μg selenium in the form of sodium selenite and 200 μg tungsten as sodium tungstate.

There was a better efficacy than without mineral intake, but less well than with encapsulation which are resistant against digestion in the small intestine, despite the larger amount.

For short-term applications for which there is no major danger that the excessive amounts of minerals accumulate in the body, it also seems that a higher dosed non-encapsulated application makes sense.

Phase N: Replacement of tungsten by molybdenum, manganese and vanadium.

In order to investigate the effectiveness of molybdenum, manganese and vanadium, first phase D was repeated, and then an administration was performed as in phase F, whereby the μg tungsten had been replaced with 20 μg Molybdenum 20 μg Vanadium and 20 μg Manganese.

There was a slightly better efficacy than without mineral intake, but much less good than with tungsten.

Phase O: Analysis of the effectiveness of indigestible carbohydrates on the retention of acetogenic bacteria population.

At the start of phase 0, acetogenic bacteria, and selenium plus tungsten were applied for 10 days analogous to phase H. Thereafter the diet was changed for 20 days to the almost fiber- and fructose-free basic diet, acetogenic bacteria were no longer applied, however, the administration of selenium and tungsten was continued. It was then converted back to the diet with fructose load, including continuation of the mineral application. A significantly higher amount of gas was measured than for example during Phases I and K.

After this phase H was again repeated for 10 days, and once more changed for 20 days to the basic diet. In addition to selenium and tungsten, however, 5 g difructose-anhydride (DFA-III) was administered distributed evenly over the day. With the subsequent conversion to the diet with fructose load again a similar small amount of gas as in phase I was able to be achieved.

The DFA III could mostly retain the population of the acetogenic bacteria during the abstention of fructose- and fiber. This is one possibility to be not always dependent on a permanent supply of acetogenic bacteria even with irregularities in the diet.

On the last day of each phase the intestinal gas volume was measured. For this a lockable double balloon colonic tube was used, which was worn for 24 hours and to which was connected a collection bag for intestinal gases and feces during the whole night and if necessary at day.

During the measurements the following quantities of gas were measured:

| | | Amount |
|---|---|---|
| | | of gas |
| Phase | Description | in liter |
| A | Basic diet | 0.25 |
| B | Diet with fructose load | 3.45 |
| C | Administration of acetogenic bacteria | 0.77 |
| D | Discontinuation of acetogenic bacteria | 3.10 |
| E | Administration of acetogenic bacteria and minerals | 0.51 |
| F | Discontinuation of acetogenic bacteria, | 2.93 |
| | continuation of the administration of minerals | |
| G | Once again administration of acetogenic bacteria | 0.70 |
| H | Additional administration of minerals coated | 0.35 |
| | resistant against digestion in the small intestine | |
| I | Discontinuation of acetogenic bacteria, | 0.42 |
| | continuation of the administration of minerals | |
| J | Discontinuation of minerals | 3.20 |
| K | Once again administration of minerals coated | 0.45 |
| | resistant against digestion in the small intestine | |
| L | Additional administration of butyrogenic bacteria | 0.30 |
| M | Non-encapsulated minerals in higher dosage | 1.45 |
| N | Replacement of tungsten by molybdenum, | 2.20 |
| | manganese and vanadium | |
| O1 | After fructose break | 0.78 |
| O2 | After fructose break with carbohydrates for | 0.48 |
| | the retention of the population | |

The measured amount of gas significantly correlated with the encountered symptoms. By the addition of acetogenic bacteria (phases C, E, G, H), a significant reduction of gas production could be achieved, which however failed to sustain after discontinuation of the administration (phases D and F). By the addition of minerals which are required for the reductive acetogenesis in a preparation coated resistant against digestion in the small intestine the gas formation could be reduced even more (phase H) and could be also maintained after the discontinuation of the administration of acetogenic bacteria (phases I and K). An addition of butyrogenic bacteria could reduce the gas formation still further (phase I). A reduction in gas formation is thus ascertained:

With the administration of the minerals and/or vitamins in a preparation coated resistant against digestion in the small intestine.

With the administration of acetogenic bacteria.

With the administration of butyrogenic bacteria.

With a combined administration of minerals in a preparation coated against digestion in the small intestine and/or vitamins, and acetogenic or butyrogenic bacteria.

With a combined administration of minerals and/or vitamins in a preparation coated agains digestion in the small intestine and butyrogenic bacteria.

With the administration in high doses of minerals, while the risks of intoxication have to be observed here.

### Example 2

The following materials are mixed in powder form:

40 μg of sodium tungstate, 30 μg sodium selenite, 50 μg manganese sulphate, 50 μg sodium molybdate, 5 mg of iron sulfate, 50 μg nickel sulfate, 50 mg yeast extract and 150 mg hydroxymethylcellulose.

Thereafter, the mixture is compressed into tablets and coated with Eudragit L 100 and Eudragit S 100 in the ratio 80:20. The thickness of the coating is adjusted, so that the ingredients are released 3 hours after reaching a pH of >6.5. Alternatively, the coating can be carried out with Eudragit FS 30 D.

An intake after at least 3 hours of soberness (4 hours after fat and protein-rich meals) and at least 30 minutes before the next ingestion (usually in the morning immediately after awakening) ensures that the active ingredients are released after transit through the small intestine.

A significant decrease of the gas formation in the colon was observed. For gas measurement a lockable double balloon colon tube was used like in example 1, which was worn for 24 hours and to which was connected a collection bag for intestinal gases and feces during the whole night and if necessary at daytime.

Furthermore, feces samples were taken from the treated patient before and after administration of the tablets. An increase in the amount of acetogenic bacteria could be detected after the initiation of the therapy and a significantly reduced gas formation with incubation of the feces sample with the addition of fructose (1 g per 100 g stool volume).

It was further noted that with an incubation of the feces without addition of nutrients, during exposure to gas consisting of 80% H2 and 20% CO2, the gas pressure decreased after the treatment, whereas it even had increased in feces samples, taken before the therapy.

### Example 3

Feces samples from 20 persons, which according to their own information did not have bloating with food typically associated with bloating (onions, cabbages, legumes), were homogenized and dispensed into 6 containers each for incubation. The incubation containers were all added with 1 g fructose per 100 g of feces, and additionally with 1. Selenium 20 μg; 2. 40 μg tungsten, 3. 20 μg Selenium plus 40 μg tungsten; 4.20 μg Selenium plus 40 μg vanadium; 5. 20 μg Selenium plus 40 μg molybdenum; 6. 40 μg Selenium plus 20 μg tungsten plus 1 drop multivitamin solution “Multibionta”, 7. 40 μg Selenium plus 20 μg tungsten plus 1 drop multivitamin solution “Multibionta” plus 1 mg of iron plus 20 μg nickel. A control tank was added only with fructose.

The incubation containers were fitted with a gas-meter, sealed gas-tight and incubated at 37°. The following amounts of gas were measured:

| | |
|---|---|
| Control | 150 ml |
| Se | 110 ml |
| W | 100 ml |
| Se + W | 20 ml |
| Se + V | 100 ml |
| Se + Mo | 85 ml |
| Se + W + Vitamin | 15 ml |
| Se + W + Vitamin + Fe + Ni | 11 ml |

With selenium and tungsten a good reduction in the amount of gas was observed, which could be still improved due to the addition of vitamins and iron plus nickel. Also tungsten or selenium alone showed significant gas reduction.

The reduction by vanadium and molybdenum was much lower on average, whereby the main influence of these minerals was achieved due to a good reduction in two of the 20 feces samples, and in the other stool samples they were almost ineffective. It is obvious that the acetogenic strains of bacteria in these stool samples used these metals in their enzymes instead of tungsten. This can be inferred from the fact that tungsten in these samples showed a less superior effectiveness. To increase the success rate, it seems advantageous to add vanadium and/or molybdenum to the tungsten.

Moreover, it is advantageous to add vitamins, iron and nickel, as this could furthermore reduce the formation of gas.

### Example 4

### Continuous Release

The following materials are mixed in powder form:

90 mg sodium tungstate, 50 mg sodium selenite, 50 mg manganese sulphate, 50 mg sodium molybdate, 5 g ferrous sulphate, 50 mg nickel sulphate, 50 g yeast extract, 50 g urea. 90% of this mixture are mixed in turn with 150 g of dibasic calcium phosphate (Encompress (R)), 20 g polyvinylpyrrolidone, 120 g hydroxypropylcellulose, and passed through a mesh 40 sieve, and granulated with 90 g hydroxypropylmethylcellulose-phthalate and 10 g beta-cyclodextrine which were dissolved together in the a sufficient quantity of acetone.

The wet pulp is dried and passed through a mesh 30 sieve to obtain the granules. Analogous to the preparations described in Example 1, acetogenic bacteria are cultured. The strains Ruminococcus hydrogenotrophicus DSM 10507, and Ruminococcus productus DSM 2950 and Ruminococcus productus DSM 3507 and Clostridium coccoides DSM 935 (Deutsche Sammlung von Mikroorganismen and Zellkulturen GmbH) are used.

The freeze-dried lyophilized bacteria of all 4 strains (each 10*10¹² colony-forming units) are mixed with the remaining 10% of the powder mixture among anaerobic conditions and filled into 1000 HPMC capsules, together with the granulate material, the capsules are closed and coated resistant against digestion in the small intestine with Eudragit FS 30 D in a thickness of 8 mg/cm². The use is carried out as in examples 1, 2, 5 to 7 or 9 to 12

### Example 5

The following materials are mixed in powder form: 40 μg of sodium tungstate, 30 μg sodium selenite and 200 mg of colloidal silicon dioxide. The mixture is filled into hydroxypropylmethylcellulose capsules and the capsules are coated with 8 mg/cm² Eudragit FS-30 D, so that they dissolve at arrival in the colon.

A woman, 28 years, normal weight, lactose intolerant with a maximum symptom free lactose tolerance of 1.5 g lactose per day (60 g cottage cheese) is treated with the coated capsules with a dosage of one capsule to each of the three main meals per day. Starting with the third day of the treatment additionally to the usually taken 1.5 g lactose per day with the diet, each main meal is enriched with lactose, starting with 0.5 g and with an increase of 0.5 g per day and main meal. After the fourth day (the 6th day since the beginning of the treatment) is being asymptomatic, the achieved daily amount of lactose (1.5 g plus 3*2 g) is maintained and the treatment is continued with empty capsules (as a placebo preparation). From the second day of the administration of placebos on, abdominal pain occurs in the form of bloating, as usually known by the subject from the period without treatment and too much lactose consumption. On the evening of the third day of the changeover to placebo, the patient asks for the allowance to cancel the test.

### Example 6

A man, 40 years, with an exocrine pancreatic insufficiency, free of symptoms with a dose of 40000 units of pancreatin per main meal (1 capsule of the pancreatin preparation Kreon 40,000 of the company Solvay) is treated with the coated capsules as in Example 5 with a dose of one capsule on each of the three main meals per day. From the third day on, the pancreatin dose is reduced (two capsules Kreon 10000 per main meal), the stool is softer, and the patient remains symptom free. From the fifth day on, the pancreatic dose is reduced again (one capsule of Kreon 10000 per main meal). The stool felt softer and slightly greasy to shiny, but not felt as inconvenient. Bloatings, which usually disturb the patient in a severe way without or with such low supply of pancreatin, continue to be absent. The expensive enzyme replacement can be significantly reduced.

### Example 7

A male subject with a small bowel resection (60% of the small intestine removed), free of symptoms with a distribution of the daily amount of food on 6 meals, is treated with the coated capsules as in Example 5 in a dose of one capsule to three main meals per day. From the third day on, the same amount of food is shifted to 5 main meals; the patient remains free of symptoms. From the fifth day on, the same amount of food is shifted to 3 meals and 2 snacks. The feces is soft and greasy shiny, but not felt as inconvenient. Bloatings, of which the patient suffered with correspondingly large meals without treatment, do not occur.

### Example 8

Due to lack of studies on the absorption of heavy metals by the colonic mucosa of infants it was not attempted to perform a treatment for the relief of the tree month colics. Since the three-month colics are caused by bloatings, which arise because the nutrients can not be absorbed entirely by the not yet completely mature small intestine of infants, and they are therefore metabolized by colonic bacteria into gases, it must be assumed, however, that also three-month colics of infants respond to the treatment with the composition of the invention. It is proposed that, after appropriate clarification of the risks, for example measurement of blood levels of such substances after application to the large intestine, beginning with harmless amounts of minerals, to treat infants with three-month colics as follows: As for the treatment of babies coated tablets are not suitable, an mixture of sodium selenite and sodium tungstate together with appropriate excipients is processed into micro-granules by methods known to the skilled person, which are then coated with a polymer that dissolves when a PH value is attained that is prevailing in the colon of babies. For this purpose for example various formulations of Eudragit are available. The application of the coating resistant to digestion in the small intestine can for instance be carried out by a fluidized bed method.

The correspondingly coated micro-granules are shaken up to form a suspension together with water, if applicable with the addition of appropriate baby food powder or breast milk to a suspension and administered during nursing bottles or breastfeeding.

### Example 9

A 25 years old man who had developed an irritable bowel syndrome after an antibiotic treatment, and every two to 3 days complaints of severe blaotings, is treated with the coated capsules analog to Example 5 in a dose of one capsule with each of the three main meals per day. During the whole testing phase of 8 days he is free of complaints.

### Example 10

A 35-year-old woman with her intestine impaired by crohn's disease is complaining of flatulence during the day (40-50 consciously perceived flatus) since the last major thrust. She is treated with the coated capsules analog Example 5 with a dose of one capsule to each of the three main meals per day. After 5 days of treatment the flatulence significantly decreases (down to 15 consciously perceived flatus during the seventh day).

### Example 11

A 42-year-old man who suffers from gluten intolerance, and a thus induced damage of the small intestinal mucosa and painful bloatings, is treated with the coated capsules analog Example 5 with a dose of one capsule of each to the three main meals per day. After 5 days of treatment a significant reduction of bloatings can be noted.

### Example 12

A 26-year-old woman with a histamine intolerance who suffers from strong bloatings after eating food rich of histamine, is treated with the coated capsules analog Example 5 in a dose of one capsule to each of the three main meals per day. After 4 days of treatment, a reduction of bloatings can be noted, after 10 days of treatment, the reduction of bloatings is clearly determined

### Example 13

Analogously to Example 5 capsules are produced, with the difference that during the filling of the agent compound into the capsule additionally freeze-dried viable bacteria of the species E. coli Nissle 1917 in an amount of 8*109 colony forming units (CFU) are added.

To a 21-year old woman who suffers from diarrhea after eating dairy products because of lactoseintolerance, one of the described capsules is administered to each main meal (3 times daily). After a length of treatment of one week due to the treatment the lactose tolerance of the patient increases from 2 g to 4 g per meal. The medication is doubled subsequently (2 capsules 3 times daily) and the tolerance limit increases to 8 g lactose per meal.

### Example 14

Analogously to Example 4 capsules are produced that are in contrast to Example 4 not coated resistant against digestion in the small intestine with Eudragit FS 30 D, but enteric-coated with Eudragit L 30 D-55.

The use is carried out analogously to Example 1, 2, 5 to 7 or 9 to 12.

### Example 15

A 36-year-old man who suffers from meteorism after eating fiber-rich meals is treated three times daily with 1.5 mg of sodium tungstate, which is dissolved in water administered with the drinking fluid during the main meals. After one week of treatment the meteorism significantly decreases.

### Example 16

A 36-year-old man who suffers from meteorism after eating fiber-rich meals is treated three times daily with 1.5 mg of sodium tungstate, which is dissolved in 100 ml water with the addition of 100 mg phytic acid and is allowed to rest for 30 minutes, and is administered to the main meals with the drinking fluid. After 4 days of treatment the meteorism significantly decreases.

1. Use of a composition comprising one or more minerals selected from the group consisting of selenium, molybdenum or tungsten, which is carried out galenically or chemically in a way that the mineral or minerals are released completely or in part, just before, during or shortly after arrival at the colon, for the manufacture of a medicament for the administration to a mammal for the prevention or reduction of gas formation in the colon and thus conditioned abdominal complaints, particularly bloatings, meteorism or abdominal cramps.

2. Use of a composition comprising tungsten and/or a tungsten compound for the administration to a mammal for the manufacture of a medicament for the prevention or reduction of gas formation in the colon and thus conditioned abdominal complaints, particularly bloatings, meteorism or abdominal cramps.

3. Use according to claim 2, characterized in that the composition is designed galenically or chemically in a way that the tungsten and/or the tungsten compound are released completely or in part, just before, during or shortly after arrival at the colon of the mammal.

4. Use according to claim 1 or 3, characterized in that the chemical implementation is realized by the bonding of the mineral or minerals to chemical compounds, which are not cleavable or absorbable in the small intestine of the mammal, preferably phytic acid, oxalic acid or tannins or tannic acids.

5. Use according to claim 1 or 3, characterized in that the galenic implementation is realized by processing, especially coating and/or grouting, of the composition with at least one pharmaceutical excipient, which is not soluble in the stomach and/or small intestine of the mammal.

6. Use according to claim 1 or 3 or 5, characterized in that the galenic implementation is designed in a way that the mineral or minerals are continuously released over a period of at least three hours, preferably up to 48 hours after passage of the stomach.

7. Use according to any preceding claim, characterized in that the composition further contains one or more strains of acetogenic bacteria.

8. Use according to claim 7, characterized in that at least one acetogenic bacterial strain is selected from the group consisting of: Ruminococcus, Eubacterium, Clostridium, or a strain isolated by a method according to claims 45 to 48

9. Use according to claim 7, characterized in that at least one of the acetogenic bacteria strains is of Ruminococcus hydrogenotrophicus, Ruminococcus productus, Eubacterium limosum, Clostridium coccoides, or Clostridium formicoaceticum.

10. Use according to any preceding claim, characterized in that the composition further contains one or more strains of butyrogenic bacteria.

11. Use according to claim 10, characterized in that at least one strain of butyrogenic bacteria is selected from the group consisting of: Fusobacterium, Faecalibacterium, Eubacterium, Clostridium, or a strain isolated by a method according to claims 41 to 44

12. Use according to any of the previous claims, characterized in that the composition also contains vanadium, nickel, iron, sodium, potassium.

13. Use according to any of the previous claims, characterized in that the composition also contains vitamins, especially vitamin A, vitamin B, vitamin C, vitamin D, vitamin E, vitamin K.

14. Use according to any preceding claim, characterized in that the composition additionally contains one or more nitrogen containing compounds and/or carbohydrate compounds, preferably poorly absorbed by the small intestine of the mammal.

15. Use according to any preceding claim, characterized in that the composition is gradually released in the colon over a period of at least 3 hours, preferably 6 hours, more preferably 12 hours.

16. Use according to any preceding claim, characterized in that the abdominal symptoms are caused by at least one of the following gastrointestinal disorders: fructose malabsorption, fructose intolerance, lactose intolerance, irritated bowel syndrome, lack of disaccharase, lack of trehalase, short bowel syndrome, irritable bowel syndrome, bloating, meteorism, diarrhea, crohn's disease, three month colic, exocrine pancreatic insufficiency, bile acid deficiency, gastric acid deficiency, dysbiosis of the intestinal flora caused by antibiotics, lack of acetogenic bacteria, lack of reductive acetogenic metabolic function of the intestinal flora, celiac disease, sprue, gluten intolerance, histamine intolerance.

17. Use according to any preceding claim, characterized in that the composition contains further enterobacteriaceae, preferably of the species Escherichia coli, either in a separate or the same pharmaceutical preparation, whereas the medicament is instead, and/or additionally designed for the treatment of diarrhea.

18. Use according to any preceding claims, characterized in that the drug instead and/or additionally earmarked to support the reconstruction and regeneration of the intestinal flora and their acetogenic metabolic function subsequent to an antibiotic treatment, hydro-colon therapy and colonic lavages or gastrointestinal infections.

19. Use according to any preceding claim, characterized in that the administration is orally or rectally.

20. Use according to any preceding claim, characterized in that the mammal is human.

21. Composition comprising one or more minerals selected from the group consisting of selenium, molybdenum or tungsten, which is carried out galenically or chemically in a way that the mineral or minerals are released completely or in part, just before, during or shortly after arrival at the colon, for the administration to a mammal for the prevention or reduction of gas formation in the colon and thus conditioned abdominal complaints, particularly bloatings, meteorism or abdominal cramps.

22. Composition comprising tungsten and/or a tungsten containing compound for the administration to a mammal for the prevention or reduction of gas formation in the colon and thus conditioned abdominal complaints, particularly bloatings, meteorism or abdominal cramps.

23. Composition according to claim 22, characterized in that it is designed galenically or chemically in a way that the tungsten and/or the tungsten compound are released completely or in part, just before, during or shortly after arrival at the colon of the mammal.

24. Composition according to claim 21 or 23, characterized in that the chemical implementation is realized by the bonding of the mineral or minerals to chemical compounds, which are not cleavable or absorbable in the small intestine of the mammal, preferably phytic acid, oxalic acid or tannins or tannic acids.

25. Composition according to claim 21 or 23, characterized in that the galenic implementation is realized by processing, especially coating and/or grouting, of the composition with at least one pharmaceutical excipient, which is not soluble in the stomach and/or small intestine of the mammal.

26. Composition according to claim 21 or 23 or 25, characterized in that the galenic implementation is designed in a way that the mineral or minerals are continuously released over a period of at least three hours, preferably up to 48 hours after passage of the stomach.

27. Composition according to one of the claims 21 to 26, characterized in that it further contains one or more strains of acetogenic bacteria.

28. Composition according to claim 27, characterized in that at least one acetogenic bacterial strain is selected from the group consisting of: Ruminococcus, Eubacterium, Clostridium, or a strain isolated by a method according to claims 45 to 48

29. Composition according to claim 27, characterized in that at least one of the acetogenic bacteria strains is of Ruminococcus hydrogenotrophicus, Ruminococcus productus, Eubacterium limosum, Clostridium coccoides, or Clostridium formicoaceticum.

30. Composition according to one of the claims 21 to 29, characterized in that the it further contains one or more strains of butyrogenic bacteria.

31. Composition according to claim 30, characterized in that at least one strain of butyrogenic bacteria is selected from the group consisting of: Fusobacterium, Faecalibacterium, Eubacterium, Clostridium, or a strain isolated by a method according to claims 41 to 44

32. Composition according to one of the claims 21 to 31, characterized in that it also contains vanadium, nickel, iron, sodium, potassium.

33. Composition according to one of the claims 21 to 32, characterized in that it also contains vitamins, especially vitamin A, vitamin B, vitamin C, vitamin D, vitamin E, vitamin K.

34. Composition according to one of the claims 21 to 33, characterized in that the composition additionally contains one or more nitrogen containing compounds and/or carbohydrate compounds, preferably poorly absorbed by the small intestine of the mammal.

35. Composition according to one of the claims 21 to 34, characterized in that it is gradually released in the colon over a period of at least 3 hours, preferably 6 hours, more preferably 12 hours.

36. Composition according to one of the claims 21 to 35, characterized in that the abdominal symptoms are caused by at least one of the following gastrointestinal disorders /: fructose malabsorption, fructose intolerance, lactose intolerance, irritated bowel syndrome, lack of disaccharase, lack of trehalase, short bowel syndrome, irritable bowel syndrome, bloating, meteorism, diarrhea, crohn's disease, three month colic, exocrine pancreatic insufficiency, bile acid deficiency, gastric acid deficiency, dysbiosis of the intestinal flora caused by antibiotics, lack of acetogenic bacteria, lack of reductive acetogenic metabolic function of the intestinal flora, celiac disease, sprue, gluten intolerance, histamine intolerance.

37. Composition according to one of the claims 21 to 36, characterized in that it contains further enterobacteriaceae, preferably of the species Escherichia coli, either in a separate or the same pharmaceutical preparation, whereas the composition is instead, and/or additionally determined for the treatment of diarrhea.

38. Composition according to one of the claims 21 to 37, characterized in that the composition instead and/or additionally determined to support the reconstruction and regeneration of the intestinal flora with acetogenic bacteria subsequent to an antibiotic treatment, hydro-colon therapy and colonic lavages or gastrointestinal infections.

39. Composition according to one of the claims 21 to 38, characterized in that it is determined to be administered orally or rectally.

40. Composition according to one of the claims 21 to 39, characterized in that the mammal is human.

41. Procedure for the isolation of butyrogenic bacteria from the feces of a mammal comprising the steps: creating a culture of bacteria from feces samples of an individual in a suitable culture medium containing as the sole energy and carbon source acetic acid.

42. Procedure according to any of the preceding claims, characterized in that the growth medium is an anaerobic nutrient medium.

43. Procedure according to any of the preceding claims, characterized in that the nutrient medium also contains an acetic acid indicator.

44. Procedure according to any of the preceding claims, characterized in that feces from asymptomatic individuals are used for the isolation.

45. Procedure for isolating acetogenic bacteria from the feces of a mammal comprising the steps: creating a culture of bacteria from feces samples of an individual in a suitable culture medium containing as the sole energy and carbon source H2 and CO2.

46. Procedure of claim 38, characterized in that the growth medium is an anaerobic nutrient medium.

47. Procedure according to any of the preceding claims, characterized in that the nutrient medium also contains an acetic acid indicator.

48. Procedure according to any of the preceding claims, characterized in that feces from asymptomatic individuals are used for the isolation.
